(19) United States
(12) Patent Application Publication
(10) Pub. No.: US 2012/0213775 A1
(43) Pub. Date: Aug. 23, 2012

(54) TREATMENT OF RENAL DISEASES

(76) Inventor: Jochen Reiser, Miami, FL (US)

(21) Appl. No.: 13/402,689

(22) Filed: Feb. 22, 2012

Related U.S. Application Data
(63) Continuation of application No. PCT/US2011/049563, filed on Aug. 29, 2011.
(60) Provisional application No. 61/377,652, filed on Aug. 27, 2010.
(60) Provisional application No. 61/381,637, filed on Sep. 10, 2010.

(51) Int. Cl. A61K 39/395; A61P 13/12; C07K 16/28

(52) U.S. Cl. 424/133.1; 424/172.1; 424/143.1; 424/142.1; 530/389.1

(57) ABSTRACT

Compositions for the treatment of renal diseases and disorders utilize agents which inhibit alphaV integrin molecules in vivo. Methods of treatment include use of these agents in the prevention and treatment of proteinuria, progressive glomerular disease and glomerular disease amongst others.

### CROSS REFERENCE TO RELATED APPLICATIONS

This application claims priority to PCT Application No. PCT/US11/49563, filed Aug. 29, 2011, which claims priority to U.S. Provisional Application No. 61/377,652, filed Aug. 27, 2010, and U.S. Provisional Application No. 61/381,637, filed Sep. 10, 2010, which is incorporated herein by reference in its entirety.

### FIELD OF THE INVENTION

Embodiments of the invention comprise compositions which prevent or treat renal diseases and methods of use.

### BACKGROUND

Integrins are a superfamily of cell adhesion receptors, which exist as heterodimeric transmembrane glycoproteins. They are part of a large family of cell adhesion receptors which are involved in cell-extracellular matrix and cell-cell interactions. Integrins play critical roles in cell adhesion to the extracellular matrix (ECM) which, in turn, mediates cell survival, proliferation and migration through intracellular signaling. The receptors consist of two subunits that are non-covalently bound. Those subunits are called alpha and beta. The alpha subunits all have some homology to each other, as do the beta subunits. The receptors always contain one alpha chain and one beta chain and are thus called heterodimeric. Both of the subunits contribute to the binding of ligand. Eighteen alpha subunits and eight beta subunits have been identified, which heterodimerize to form at least 24 distinct integrin receptors.

Among the variety of alpha chain subunits is a protein chain referred to as alpha V. The ITAGV gene encodes integrin alpha chain V (alphaV). The I-domain containing integrin alpha V undergoes post-translational cleavage to yield disulfide-linked heavy and light chains, that combine with multiple integrin beta chains to four different integrins. Alternative splicing of the gene yields 7 different transcripts; a, b, c, e, f, h, j altogether encoding 6 different protein isoforms of alphaV. Among the known associating beta chains (beta chains 1, 3, 5, 6, and 8; ‘ITGB1’, ‘ITGB3’, ‘ITGB5’, ‘ITGB6’, and ‘ITGB8’), each can interact with extracellular matrix ligands. The alpha V beta 3 integrin, perhaps the most studied of these, is referred to as the vitronectin receptor (VNR). In addition to providing for cell attachment to other cells or to extracellular proteins such as vitronectin (alphaVbeta3) and fibronectin (alphaVbeta6), the integrins are capable of intracellular signaling which provides clues for cell migration and secretion of or elaboration of other proteins involved in cell motility and invasion and angiogenesis. The alpha V integrin subfamily of integrins recognize the ligand motif Arg-GlyAsp (RGD) present in fibronectin, vitronectin, Von Willebrand factor, and fibrinogen.

### SUMMARY

This Summary is provided to present a summary of the invention to briefly indicate the nature and substance of the invention. It is submitted with the understanding that it will not be used to interpret or limit the scope or meaning of the claims.

Embodiments of the invention are directed to compositions for the treatment renal diseases or disorders, such as for example, proteinuria.

Other aspects are described infra.

### BRIEF DESCRIPTION OF THE DRAWINGS

FIG. 1 is a schematic outlining a normal filtration barrier (a) and an impaired barrier (b) in glomerular disease that is characterized by foot process effacement. On a mechanistic level, the urokinase receptor can associate with podocyte integrins (in particular beta3 integrins) and increase integrin activity. This step is critical for foot process effacement and the development of proteinuric kidney disease.

FIG. 2 is a graph showing the treatment of kidney disease. CNTO95 was administered in escalating dosages (ranging from 10 mg/kg to 100 mg/kg) intravenously (for small dosages, the injection volume was topped up to 500 μl with PBS; for large dosages when the CNTO95 volume is more than 500 μl, the actual required volume was injected). The first injection of CNTO95 was given 1 hour prior to the injection of Puromycin aminonucleoside (PAN). Additional injections were given on day 2, 4, 6, 8, 12, and 21. 8 days after induction of proteinuric kidney disease by PAN, there was a significant reduction of proteinuria by up to 27% (p<0.05). The day 8 timepoint is considered the peak phase of proteinuria. CNTO95 is only poorly reactive in rats (please see Kd values) yet still is associated with a significant reduction in proteinuria.

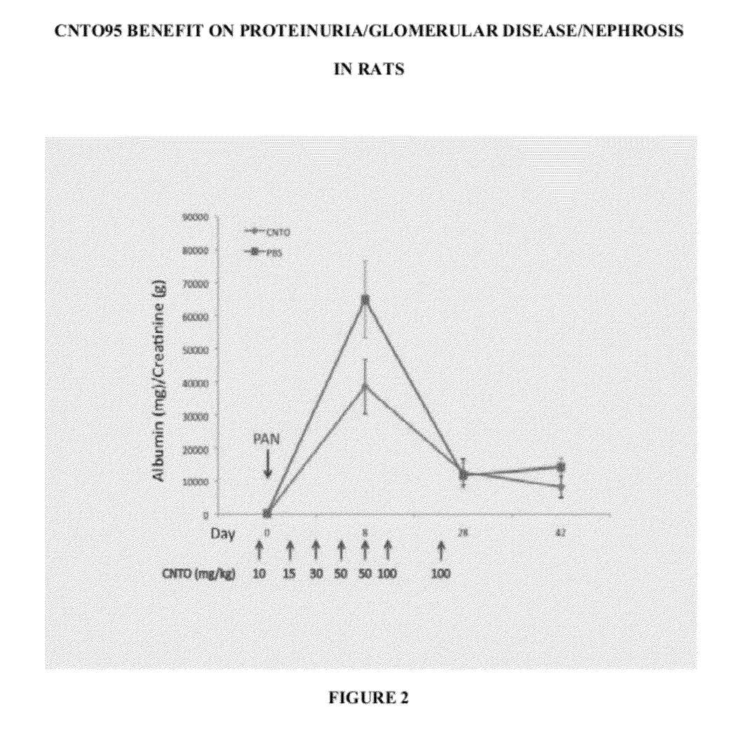

FIG. 3 is a graph showing CNTO95 specific for the alphaVbeta3 integrin reduces proteinuria in PAN rats. CNTO95 was administered intravenously at a volume of 500 μl 1 hour prior to the injection of Puromycin aminonucleoside. This set up is a preventive set-up. 8 days after induction of proteinuric kidney disease, there was a significant reduction of proteinuria by up to 27% (p<0.05). The 7-8 day timepoint is considered the peak phase of proteinuria. CNTO95 is only poorly reactive in rats (please see K_{d} values) yet still is associated with a significant reduction in proteinuria.

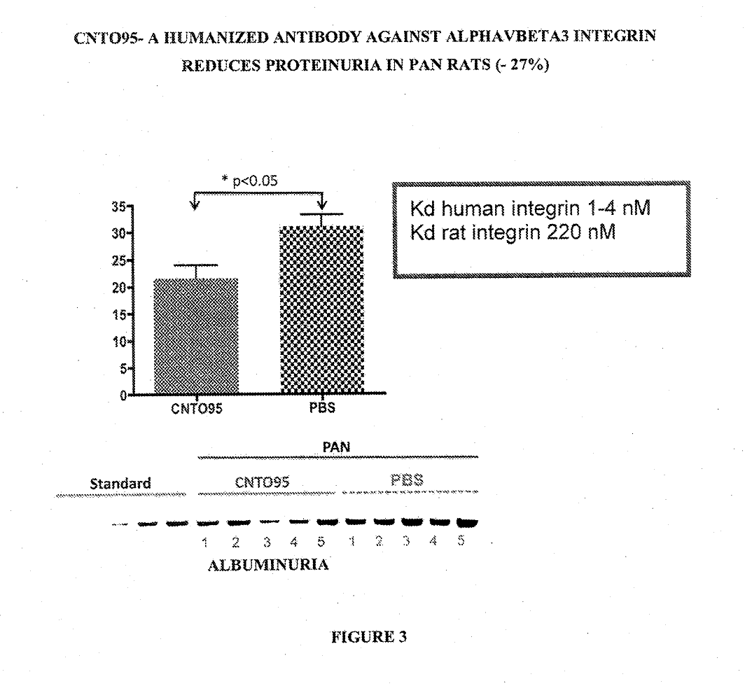

FIG. 4 is a graph showing baseline proteinuria of rats before receiving CNTO95 and/or PAN. Rats show comparable levels of minimal baseline proteinuria (left panel). CNTO95 was administered intravenously at a volume of 500 μl 1 hour prior to the injection of Puromycin aminonucleoside. This set up is a preventive set-up. 8 days after induction of proteinuric kidney disease, there was a significant reduction of proteinuria by up to 27%. The day 8 timepoint is considered the peak phase of proteinuria. CNTO95 is only poorly reactive in rats (please see K_{d} values) yet still is associated with a significant reduction in proteinuria.

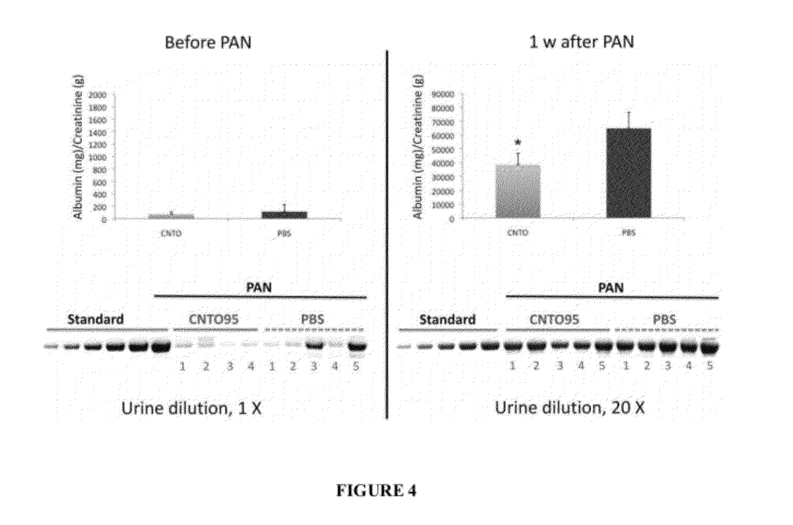

FIG. 5 is a graph showing the effects of CNTO95 administration 6 weeks after PAN induced glomerular proteinuria. A 38% reduction of proteinuria (p<0.05) was noted. Furthermore, one rat died in the non-CNTO95 group.

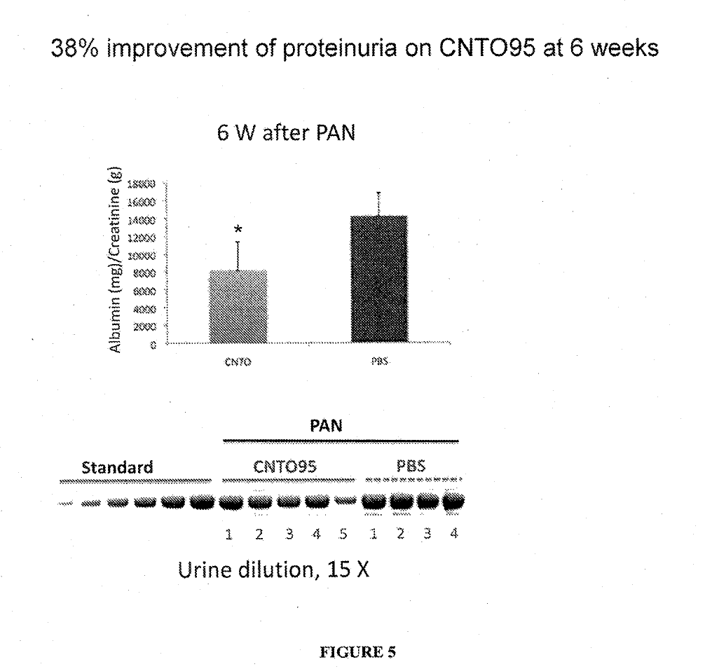

FIG. 6 shows the results of immunofluorescence after incubation of CNTO95 with differentiated human podocytes in cell culture model. The staining in green comes from immunofluorescent labeling of active beta3 integrins using AP5 antibody. Under control conditions, there is a low baseline AP5 labeling. It is however much increased after 24 hours of PAN treatment (see also Wei et al. Nat. Med. 2008). PAN was given 4 hours prior to CNTO95 (treatment approach) and then left active for another 20 hours (together with CNTO95 at 1 microgram/ml). A reduction of AP5 signal was noted indicating reduction in beta3 integrins (such as alphavbeta3 or alphavbeta5). The middle panel shows human podocytes treated with different dosages of CNTO95 from 1 μg/ml to 10 μg/ml. AP5 labeling starts to increase at high dosages of CNTO95 which is most likely due to clustering of beta3 integrins induced by CNTO95 in high concentration. Lower panel: Podocytes under normal conditions, and after stimulation with soluble uPAR as well as treated with soluble uPAR plus CNTO95 (1 microgram/ml) were compared for AP5 labeling. suPAR induced the AP5 label but not in the presence of CNTO95 (1 microgram/ml).

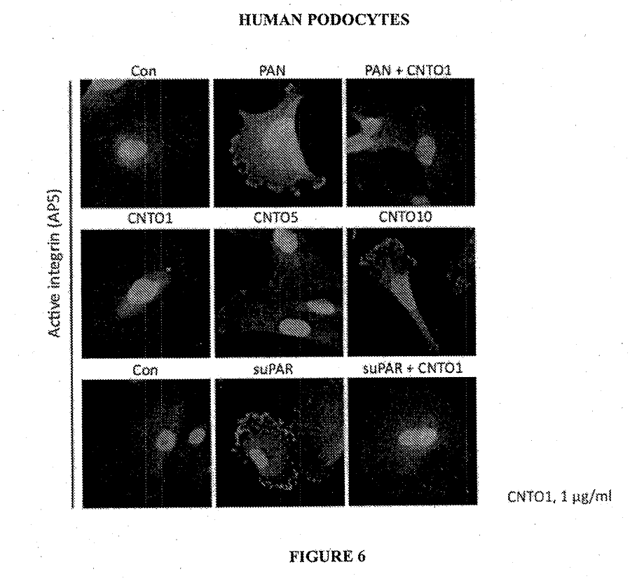

FIG. 7 is a scan of a photograph of an immunostain showing the activity of beta3 integrin in podocytes (using AP5 antibody) in human Diabetic Nephropathy stages CKD 2-4.

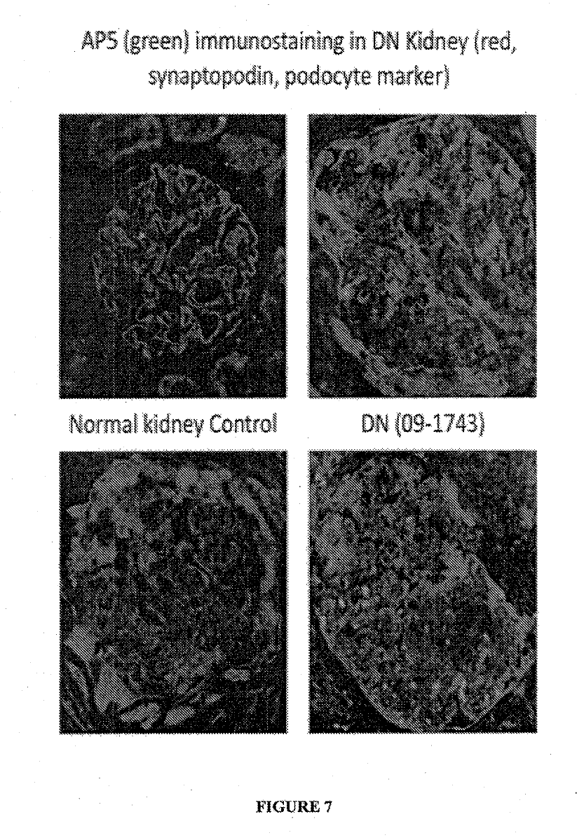

### DETAILED DESCRIPTION

Embodiments of the invention relate to discoveries involving agents which modulate and/or inhibit the function, expression, activity or combinations thereof, of alphaV (aV) integrins. Modulation of the alpha V integrins are directed to treatment of kidney diseases or disorders such as, for example, proteinuria.

Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention.

All genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes disclosed herein, which in some embodiments relate to mammalian nucleic acid and amino acid sequences are intended to encompass homologous and/or orthologous genes and gene products from other animals including, but not limited to other mammals, fish, amphibians, reptiles, and birds. In preferred embodiments, the genes or nucleic acid sequences are human.

### DEFINITIONS

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “safe and effective amount” or “therapeutic amount” refers to the quantity of a component which is sufficient to yield a desired therapeutic response without undue adverse side effects (such as toxicity, irritation, or allergic response) commensurate with a reasonable benefit/risk ratio when used in the manner of this invention. By “therapeutically effective amount” is meant an amount of a compound of the present invention effective to yield the desired therapeutic response. The specific safe and effective amount or therapeutically effective amount will vary with such factors as the particular condition being treated, the physical condition of the patient, the type of mammal or animal being treated, the duration of the treatment, the nature of concurrent therapy (if any), and the specific formulations employed and the structure of the compounds or its derivatives.

As used herein “proteinuria” refers to any amount of protein passing through a podocyte that has suffered podocyte damage or through a podocyte mediated barrier that normally would not allow for any protein passage. In an in vivo system the term “proteinuria” refers to the presence of excessive amounts of serum protein in the urine. Proteinuria is a characteristic symptom of either renal (kidney), urinary, pancreatic distress, nephrotic syndromes (i.e., proteinuria larger than 3.5 grams per day), eclampsia, toxic lesions of kidneys, and it is frequently a symptom of diabetes mellitus. With severe proteinuria general hypoproteinemia can develop and it results in diminished oncotic pressure (ascites, edema, hydrothorax).

As used herein, the terms “podocyte disease(s)” and “podocyte disorder(s)” are interchangeable and mean any disease, disorder, syndrome, anomaly, pathology, or abnormal condition of the podocytes or of the structure or function of their constituent parts.

The phrase “specifically binds to”, “is specific for” or “specifically immunoreactive with”, when referring to an antibody refers to a binding reaction which is determinative of the presence of the protein in the presence of a heterogeneous population of proteins and other biologics. For example, an antibody “specifically binds” or “preferentially binds” to a target or epitope if it binds with greater affinity, avidity, more readily, and/or with greater duration than it binds to other targets. Thus, under designated immunoassay conditions, the specified antibodies bind to a particular protein and do not bind in a significant amount to other proteins present in the sample. Specific binding to a protein under such conditions may require an antibody that is selected for its specificity for a particular protein.

The terms “detecting”, “detect”, “identifying”, “quantifying”, “measuring” includes assaying, quantitating, imaging or otherwise establishing the presence or absence of the urinary proteins or other disease indicators, and the like, or assaying for, imaging, ascertaining, establishing, or otherwise determining the prognosis and/or diagnosis of renal diseases, disorders or conditions.

“Patient” or “subject” refers to mammals and includes human and veterinary subjects.

As used herein “a patient in need thereof’ refers to any patient that is affected with a disorder characterized by proteinuria. In one aspect of the invention “a patient in need thereof refers to any patient that may have, or is at risk of having a disorder characterized by proteinuria.

As used herein, the terms “test substance” or “candidate therapeutic agent” or “agent” are used interchangeably herein, and the terms are meant to encompass any molecule, chemical entity, composition, drug, therapeutic agent, chemotherapeutic agent, or biological agent capable of preventing, ameliorating, or treating a disease or other medical condition. The term includes small molecule compounds, antisense reagents, siRNA reagents, antibodies, enzymes, peptides organic or inorganic molecules, natural or synthetic compounds and the like. A test substance or agent can be assayed in accordance with the methods of the invention at any stage during clinical trials, during pre-trial testing, or following FDA-approval.

As used herein the phrase “diagnostic” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

As used herein the phrase “diagnosing” refers to classifying a disease or a symptom, determining a severity of the disease, monitoring disease progression, forecasting an outcome of a disease and/or prospects of recovery. The term “detecting” may also optionally encompass any of the above. Diagnosis of a disease according to the present invention can be effected by determining a level of a polynucleotide or a polypeptide of the present invention in a biological sample obtained from the subject, wherein the level determined can be correlated with predisposition to, or presence or absence of the disease. It should be noted that a “biological sample obtained from the subject” may also optionally comprise a sample that has not been physically removed from the subject, as described in greater detail below.

As defined herein, “a therapeutically effective amount” of an agent or compound (i.e., an effective dosage) means an amount sufficient to produce a therapeutically (e.g., clinically) desirable result. The compositions can be administered one from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds of the invention can include a single treatment or a series of treatments. A “prophylactically effective amount” may refer to the amount of an agent sufficient to prevent the recurrence or spread of kidney diseases or disorders, particularly proteinuria, or the occurrence of such in a patient, including but not limited to those predisposed to kidney disease, for example those genetically predisposed to kidney disease or previously exposed to environmental factors, such as for example, alcohol or infectious organisms such as hepatitis virus. A prophylactically effective amount may also refer to the amount of the prophylactic agent that provides a prophylactic benefit in the prevention of disease. Further, a prophylactically effective amount with respect to an agent of the invention means that amount of agent alone, or in combination with other agents, that provides a prophylactic benefit in the prevention of disease.

The term “sample” is meant to be interpreted in its broadest sense. A “sample” refers to a biological sample, such as, for example; one or more cells, tissues, or fluids (including, without limitation, plasma, serum, whole blood, cerebrospinal fluid, lymph, tears, urine, saliva, milk, pus, and tissue exudates and secretions) isolated from an individual or from cell culture constituents, as well as samples obtained from, for example, a laboratory procedure. A biological sample may comprise chromosomes isolated from cells (e.g., a spread of metaphase chromosomes), organelles or membranes isolated from cells, whole cells or tissues, nucleic acid such as genomic DNA in solution or bound to a solid support such as for Southern analysis, RNA in solution or bound to a solid support such as for Northern analysis, cDNA in solution or bound to a solid support, oligonucleotides in solution or bound to a solid support, polypeptides or peptides in solution or bound to a solid support, a tissue, a tissue print and the like.

Numerous well known tissue or fluid collection methods can be utilized to collect the biological sample from the subject in order to determine the level of DNA, RNA and/or polypeptide of the variant of interest in the subject. Examples include, but are not limited to, fine needle biopsy, needle biopsy, core needle biopsy and surgical biopsy (e.g., brain biopsy), and lavage. Regardless of the procedure employed, once a biopsy/sample is obtained the level of the variant can be determined and a diagnosis can thus be made.

The term “urokinase receptor molecule”, “uPAR” is meant to include, soluble, membrane bound, variants, fragments, all family members, isoforms, precursors, mutants, alleles, fragments, species, sense and antisense polynucleotide strands, etc.

The term “neutralizing” when referring to an targeted binding agent such as an antibody relates to the ability of an antibody to eliminate, or significantly reduce, the activity of a target antigen. Accordingly, a “neutralizing” anti-uPAR antibody of the invention is capable of eliminating or significantly reducing the activity of uPAR. A neutralizing uPAR antibody may, for example, act by blocking the binding of uPA to its receptor uPAR. By blocking this binding, the uPA mediated plasminogen activation is significantly, or completely, eliminated.

“Active” or “activity” in regard to a uPAR polypeptide refers to a portion of an uPAR polypeptide that has a biological or an immunological activity of a native uPAR polypeptide. “Biological” when used herein refers to a biological function that results from the activity of the native uPAR polypeptide.

As used herein, the terms “antibody” and “antibodies” refer to monoclonal antibodies, multispecific antibodies, human antibodies, humanized antibodies, synthetic antibodies, chimeric antibodies, polyclonal antibodies, camelized antibodies, single-chain Fvs (scFv), single chain antibodies, Fab fragments, F(ab′) fragments, disulfide-linked bispecific Fvs (sdFv), intrabodies, and anti-idiotypic (anti-Id) antibodies (including, e.g., anti-Id and anti-anti-Id antibodies to antibodies of the invention), and epitope-binding fragments of any of the above. In particular, antibodies include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgG₁, IgG₂, IgG₃, IgG₄, IgA₁ and IgA₂) or subclass.

Antibodies of the present invention can be in any of a variety of forms, including whole immunoglobulins, antibody fragments, single chain antibodies which includes the variable domain complementarity determining regions (CDR), and the like forms, all of which fall under the broad term “antibody”, as used herein.

The term “antigen binding fragment” refers to an antibody fragment or portion of a full-length antibody, generally the variable region. Examples of antigen binding fragments fragments of an antibody include Fab, Fab′, F(ab′)₂ and Fv fragments. Papain digestion of antibodies produces two identical antigen binding fragments, called the Fab fragment, each with a single antigen binding site, and a residual “Fc” fragment, so-called for its ability to crystallize readily. Pepsin treatment yields an F(ab′)₂ fragment that has two antigen binding fragments that are capable of cross-linking antigen. Additional fragments can include diabodies, linear antibodies, single-chain antibody molecules, and multispecific antibodies formed from antibody fragments.

Single chain antibody (“SCA”), defined as a genetically engineered molecule containing the variable region of the light chain, the variable region of the heavy chain, linked by a suitable polypeptide linker as a genetically fused single chain molecule. Such single chain antibodies are also referred to as “single-chain Fv” or “sFv” antibody fragments. Generally, the Fv polypeptide further comprises a polypeptide linker between the VH and VL domains that enables the sFv to form the desired structure for antigen binding. For a review of sFv see Pluckthun in The Pharmacology of Monoclonal Antibodies 113: 269-315 Rosenburg and Moore eds. Springer-Verlag, NY1 1994. Methods for producing sFvs are described, for example, by Whitlow, et al., 1991, In: Methods: A Companion to Methods in Enzymology, 2:97;

Another form of an antibody fragment is a peptide coding for a single complementarity-determining region (CDR). CDR peptides (“minimal recognition units”) are often involved in antigen recognition and binding. CDR peptides can be obtained by cloning or constructing genes encoding the CDR of an antibody of interest. Such genes are prepared, for example, by using the polymerase chain reaction to synthesize the variable region from RNA of antibody-producing cells. See, for example, Larrick, et al., Methods: a Companion to Methods in Enzymology, Vol. 2, page 106 (1991).

The term “diabodies” refers to a small antibody fragments with two antigen-binding sites, which fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, e.g., EP 404,097; or Hollinger et al., Proc. Natl. Acad. Sci. USA 90: 6444-6448 (1993).

In accordance with the present invention, there may be employed conventional molecular biology, microbiology, recombinant DNA, immunology, cell biology and other related techniques within the skill of the art. See, e.g., Sambrook et al., (2001) Molecular Cloning: A Laboratory Manual. 3rd ed. Cold Spring Harbor Laboratory Press: Cold Spring Harbor, N.Y.; Sambrook et al., (1989) Molecular Cloning: A Laboratory Manual. 2nd ed. Cold Spring Harbor Laboratory Press: Cold Spring Harbor, N.Y.; Ausubel et al., eds. (2005) Current Protocols in Molecular Biology. John Wiley and Sons, Inc.: Hoboken, N.J.; Bonifacino et al., eds. (2005) Current Protocols in Cell Biology. John Wiley and Sons, Inc.: Hoboken, N.J.; Coligan et al., eds. (2005) Current Protocols in Immunology, John Wiley and Sons, Inc.: Hoboken, N.J.; Coico et al., eds. (2005) Current Protocols in Microbiology, John Wiley and Sons, Inc.: Hoboken, N.J.; Coligan et al., eds. (2005) Current Protocols in Protein Science, John Wiley and Sons, Inc.: Hoboken, N.J.; Enna et al., eds. (2005) Current Protocols in Pharmacology John Wiley and Sons, Inc.: Hoboken, N.J.; Hames et al., eds. (1999) Protein Expression: A Practical Approach. Oxford University Press: Oxford; Freshney (2000) Culture of Animal Cells: A Manual of Basic Technique. 4th ed. Wiley-Liss; among others. The Current Protocols listed above are updated several times every year.

### Compositions

Proteinuria can be primarily caused by alterations of structural proteins involved in the cellular mechanism of filtration. The pathophysiological causes of proteinuria can be divided in the following major groups: (1) genetically determined disturbances of the structures which form the “glomerular filtration unit” like the glomerular basement membrane, the podocytes, or the slit diaphragm, (2) inflammatory processes, either directly caused by autoimmune processes or indirectly induced by microbes, (3) damage,of the glomeruli caused by agents, or (4) as the final result of progressive tubulointerstitial injury finally resulting in the loss of function of the entire nephron.

The central metabolism of a cell can determine its short- and long-term structure and function. When a disease state arises, the metabolism (i.e., the transportation of nutrients into the cells, the overall substrate utilization and production, synthesis and accumulation of intracellular metabolites, etc.) is altered in a way that may permit the cell to adapt under the changing physiologic constraints. Diabetes mellitus is a metabolic disease that also affects podocytes, key cells that regulate glomerular filtration. A pathological role for a cytoplasmic variant of cathepsin L enzyme as the biological instigator of kidney filter dysfunction (proteinuria) and progression of renal disease through cleavage of different types of critical podocyte target proteins. Podocytes are highly differentiated cells that reside in the kidney glomeruli. Their foot processes (FP) and interposed slit diaphragm (SD) form the final barrier to protein loss. Podocyte injury is typically associated with FP effacement and urinary protein loss.

In a healthy person, urinary protein excretion is less than 150 mg/day and consists mainly of filtered plasma proteins (60%) and tubular Tamm-Horsfall proteins (40%). The main plasma protein in the urine is albumin, constituting about 20% of daily protein excretion. In healthy subjects, the daily amount of urinary albumin is less than 20 mg (13.8 mg/min). Proteinuria usually reflects an increase in glomerular permeability for albumin and other plasma macromolecules. A 24-h urine collection containing more than 150 mg of protein is considered pathological. There are several basic types of proteinuria; for example, glomerular, tubular, overflow, and exercise-induced. Glomerular proteinuria is the most common form (around 90%). Low molecular weight molecules, such as μ2-microglobulin, amino acids, and immunoglobulin light chains, have a molecular weight of about 25 kDa (albumin is 69 kDa). These smaller proteins are readily filtered across the glomerular filtration barrier and then fully reabsorbed by the proximal tubule. A variety of diseases that affect tubular and interstitial cell integrity impair the tubular reabsorption of these molecules. Some forms of glomerular diseases are also accompanied by tubular injury and tubular proteinuria.

Pathological processes, such as multiple myeloma with a production of paraproteins, can result in increased excretion of low molecular weight proteins into the urine, a process termed overflow proteinuria. In this scenario, proteinuria results from the amount of filtered proteins exceeding the reabsorptive capacity of the proximal tubule. Dynamic exercise can also result in increased urinary excretion of proteins, predominantly of plasma origin, during and following physical exercise. A number of terms have been used to describe this phenomenon-post-exercise proteinuria, athletic pseudonephritis, exercise proteinuria, or exercise-induced proteinuria. Maximal rates of proteinuria occur approximately 30 min after exercise, with a resolution toward resting levels within 24-48 h. The magnitude of proteinuria varies from near normal to heavy (47 g/day), with the greatest levels up to 100 times that of rest observed after high-intensity exercise, such as a marathon. It is noteworthy that post-exercise proteinuria is transient in nature and not associated with any particular renal disease, raising the intriguing possibility that at least some forms of proteinuria (e.g., post-exercise, post-prandial, infection-associated) may reflect a normal, physiological response of the human body.

Embodiments of the invention are directed to inhibiting both soluble and membrane bound forms of urokinase receptor activation of alpha V integrins. Both soluble as well as podocyte-membrane bound forms of urokinase receptor can activate integrin alphaVbeta₃ (αVβ₃) as well as integrin alphaVbeta₅ (αVβ₅) in podocytes and cause renal disease. Urokinase receptor (uPAR) signaling in podocytes has been recently shown to cause glomerular disease. The soluble form of the urokinase receptor (suPAR) can be deposited in the kidney and cause proteinuric renal disease. Like endogenous podocyte uPAR, suPAR activates αᵥβ₃ integrin in an outside in dependent fashion. uPAR is a glycosylphosphatidylinositol (GPI)-anchored protein with three extracellular domains. Cleavage of the GPI anchor generates suPAR. suPAR has been found elevated in sera of patients with HIV, rheumatic or neurological diseases, hematologic malignancies and epithelial tumors. Proteinuria caused by uPAR-β3-integrin signaling can be prevented and reduced by cyclo-RGDfV, a selective inhibitor of αVβ₃-integrin.

In a preferred embodiment, a composition comprises an agent which specifically binds to αVβ3 and/or αVβ5 integrins and modulates expression, function, signaling or combinations thereof.

In one preferred embodiment the composition comprises an agent which modulates αVβ3 and/or αVβ5 integrin signaling. In preferred embodiments, the agent selectively inhibits urokinase receptor-β3 (UPAR-β3) and urokinase receptor-β5 (UPAR-β5) integrin mediated signaling. Embodiments also include the soluble urokinase receptor-β3 (suPAR-β3) and urokinase receptor-β5 (suPAR-β5) integrin mediated signaling.

In another preferred embodiment, an agent which specifically binds to integrins αVβ3 and/or αVβ5 and modulates expression, function, signaling or combinations thereof, comprises an antibody, aptamer, small molecule, peptide, polypeptide, oligonucleotide, polynucleotide, enzymes, synthetic molecules, organic or inorganic molecules.

In another preferred embodiment, an agent which specifically binds to integrins αVβ₃ and/or αVβ5 and modulates signaling mediated events by these integrins is an antibody. VITAXIN® (etaracizumab) is an example of an antibody that specifically binds to integrin αVβ3. An example of an antibody which specifically binds to integrins αVβ3 and αVβ5 is CNTO 95 (Trikha M, et al., Int J. Cancer. 2004 Jun. 20; 110(3):326-35). CNTO 95 is a fully human antibody that recognizes the alphaV family of integrins and is to be less immunogenic in humans compared to chimeric or humanized antibodies. CNTO 95 bound to purified αVβ3 and αVβ5 with a Kd of approximately 200 μM and to alphaV integrin-expressing human cells with a Kd of 1-24 nM. In vitro, CNTO 95 inhibited human melanoma cell adhesion, migration and invasion at doses ranging 7-20 nM. (Trikha M, et al., Int J. Cancer. 2004 Jun. 20; 110(3):326-35). Other preferred antibodies which specifically bind to integrins αVβ3 and/or αVβ5 include those having one or more (e.g., 1, 2, 3, 4, 5, or 6) of the complementarity determining regions of CNTO 95 or etaracizumab.

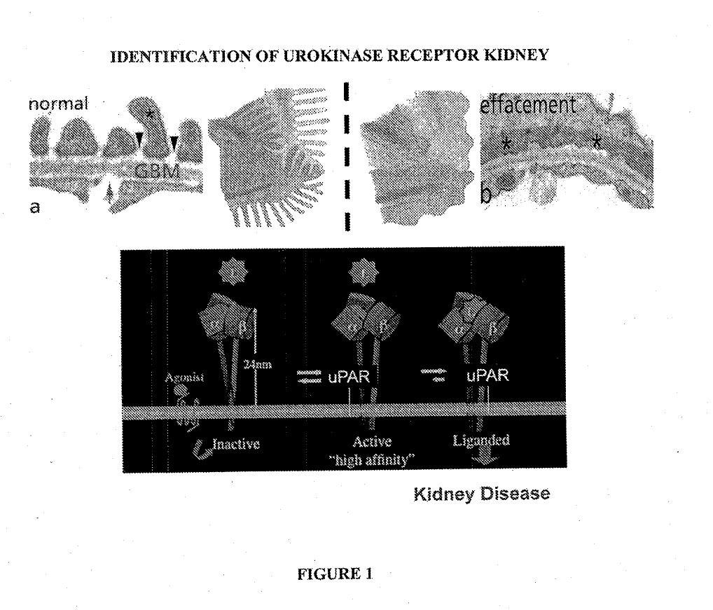

In the studies, herein, the antibody was used to treat glomerular kidney disease. Briefly, nephrosis was induced in rats by a single injection of puromycin aminonucleoside (PAN) i.p. Two groups of rats were formed. Group A (n=5) received only PAN, whereas the other group received PAN plus escalating doses of CNTO 95 on days 1, 3, 5 and 7 before urine was analyzed on day 8 (FIG. 1). Day 8 represents the peak time point for proteinuria in this model. An approximate 27% reduction in proteinuria was observed at this time. It is anticipated that CNTO 95 has about fifty (50) fold more potency in humans over rat and thus a 27% reduction of proteinuria in rats represents an excellent result.

In another preferred embodiment, a method of preventing or treating kidney disease in vivo, comprises administering to a patient an agent in a therapeutically effective amount, whereby the agent modulates the expression, function or signaling of alphaV integrin molecules in vivo. The agent is specific for binding to alpha V integrins and modulates the expression, blocking the active binding site by molecules, such as, the soluble and membrane bound forms of the urokinase receptor, the activities or functions of alpha V integrin molecules, such as for example, cell-to-cell interactions, inter- and intra-cellular signaling and the like.

In another preferred embodiment, the agent comprises an antibody, aptamer, small molecule, enzyme, oligonucleotide, polynucleotide, peptide, polypeptide, synthetic molecule, organic or inorganic molecule.

In another preferred embodiment, the agent specifically binds to alphaVbeta3 (αVβ3) and alphaVbeta5 (αVβ5) integrins.

In a preferred embodiment, the agent modulates or inhibits alphaV integrin molecules expression, function and/or activity by about 5% as compared to a normal control, preferably by about 10%, preferably by about 50%, preferably by about 80%, 90%, 100%. Modulation of the, for example, soluble urokinase receptor molecules expression or amounts results in for example, a decrease in aV integrin activation and treatment of renal diseases such as proteinuria.

In another preferred embodiment an agent inhibits or blocks activated uPAR-β3-integrin signaling and uPAR-β5-integrin signaling and podocyte FP hypermotility.

In another preferred embodiment, the composition comprises one or more agents which modulate αV integrin expression, activity, and/or function in vivo. For example, one agent directly inhibits αV integrin activity. In another example, an agent directly inhibits binding of uPAR to αV integrins or associated molecules which result in changes to αV integrin signaling. In another preferred embodiment, a mimetic of αVβ3 and αVβ5 ligand inhibits αV activity or functions.

In another preferred embodiment, a combination of agents which modulate αVβ3 and/or αVβ5 expression, function and/or activity on are administered to a patient, for example, in the treatment of a disease or disorder characterized by proteinuria and/or podocyte diseases or disorders.

In a preferred embodiment, a disease or disorder characterized by proteinuria comprises: glomerular diseases, membranous glomerulonephritis, focal segmental glomerulonephritis, minimal change disease, nephrotic syndromes, pre-eclampsia, eclampsia, kidney lesions, collagen vascular diseases, stress, strenuous exercise, benign orthostatic (postural) proteinuria, focal segmental glomerulosclerosis (FSGS), IgA nephropathy, IgM nephropathy, membranoproliferative glomerulonephritis, membranous nephropathy, sarcoidosis, Alport's syndrome, diabetes mellitus, kidney damage due to drugs, Fabry's disease, infections, aminoaciduria, Fanconi syndrome, hypertensive nephrosclerosis, interstitial nephritis, Sickle cell disease, hemoglobinuria, multiple myeloma, myoglobinuria, cancer, Wegener's Granulomatosis or Glycogen Storage Disease Type 1.

In another preferred embodiment, an agent which modulates αVβ3 and αVβ5 integrin signaling is administered to patients suffering from or pre-disposed to developing a podocyte disease or disorder. Podocyte diseases or disorders include but are not limited to loss of podocytes (podocytopenia), podocyte mutation, an increase in foot process width, or a decrease in slit diaphragm length. In one aspect, the podocyte-related disease or disorder can be effacement or a diminution of podocyte density. In one aspect, the diminution of podocyte density could be due to a decrease in a podocyte number, for example, due to apoptosis, detachment, lack of proliferation, DNA damage or hypertrophy.

In one embodiment, the podocyte-related disease or disorder can be due to a podocyte injury. In one aspect, the podocyte injury can be due to mechanical stress such as high blood pressure, hypertension, or ischemia, lack of oxygen supply, a toxic substance, an endocrinologic disorder, an infection, a contrast agent, a mechanical trauma, a cytotoxic agent (cis-platinum, adriamycin, puromycin), calcineurin inhibitors, an inflammation (e.g., due to an infection, a trauma, anoxia, obstruction, or ischemia), radiation, an infection (e.g., bacterial, fungal, or viral), a dysfunction of the immune system (e.g., an autoimmune disease, a systemic disease, or IgA nephropathy), a genetic disorder, a medication (e.g., anti-bacterial agent, anti-viral agent, anti-fungal agent, immunosuppressive agent, anti-inflammatory agent, analgesic or anticancer agent), an organ failure, an organ transplantation, or uropathy. In one aspect, ischemia can be sickle-cell anemia, thrombosis, transplantation, obstruction, shock or blood loss. In one aspect, the genetic disorders may include congenital nephritic syndrome of the Finnish type, the fetal membranous nephropathy or mutations in podocyte-specific proteins, such as α-actin-4, podocin and TRPC6.

In one aspect, the podocyte-related disease or disorder can be an abnormal expression or function of slit diaphragm proteins such as podocin, nephrin, CD2AP, cell membrane proteins such as TRPC6, and proteins involved in organization of the cytoskeleton such as synaptopodin, actin binding proteins, lamb-families and collagens. In another aspect, the podocyte-related disease or disorder can be related to a disturbance of the GBM, to a disturbance of the mesangial cell function, and to deposition of antigen-antibody complexes and anti-podocyte antibodies. In another aspect, the podocyte-related disease or disorder can be tubular atrophy.

In a preferred embodiment, the podocyte-related disease or disorder comprises proteinuria, such as microalbumiuria or macroalbumiuria. Thus, in some preferred embodiments, one or more agents which modulate αV integrin expression, function, activity, can be combined with one or more other chemotherapeutic compounds which are used to treat any of the podocyte diseases or disorders.

In another preferred embodiment, a method of preventing or treating progressive glomerular disease comprises an agent which modulates αV integrin expression, function, activity, and decreases proteinuria levels to a clinically normal level. Proteinuria values or levels can be measured by any typical assay, diagnostic or otherwise.

A wide variety of agents can be used to target αVβ3 and αVβ5 integrins. These agents may be designed to target signaling by having an in vivo activity which reduces the expression and/or activity of αV and associated molecules. For example, the agents may regulate αV molecules based on the cDNA or regulatory regions, using for example, DNA-based agents, such as antisense inhibitors and ribozymes, can be utilized to target both the introns and exons of the αV genes as well as at the RNA level.

Alternatively, the agents may target αV molecules based on the amino acid sequences including the propieces and/or three-dimensional protein structures of αV molecules. Protein-based agents, such as human antibody, non-human monoclonal antibody and humanized antibody, can be used to specifically target different epitopes on αVβ3 and αVβ5 molecules. Peptides or peptidomimetics can serve as high affinity inhibitors to specifically bind to the active site of αVβ3 and αVβ5, inhibiting the in vivo activity of the αVβ3 and αVβ5, such as for example, signaling. Small molecules may also be employed.

In addition to targeting αV molecules, agents may also be used which competitively inhibit αV molecules by competing with the natural ligands of αVβ3 and αVβ5.

Antibodies: In other embodiments of the invention described herein relate to targeted binding agents that bind αV integrins and affect αV function. Examples include, monoclonal antibodies that bind αVβ3 and αVβ5 integrins and affect their function.

In another preferred embodiment, the invention relates to fully human anti-αV antibodies which bind to both αVβ3 and αVβ5 integrins with desirable properties from a therapeutic perspective, including high binding affinity for αVβ3 and αVβ5 integrins in vitro and in vivo.

In one embodiment, the invention includes antibodies that bind to αVβ3 and αVβ5 integrins with very high affinities (Kd). For example a human, rabbit, mouse, chimeric or humanized antibody that is capable of binding αVβ3 and αVβ5 integrins with a Kd less than, but not limited to, 10⁻⁵, 10⁻⁶, 10⁻⁷, 10⁻⁸, 10⁻⁹, 10⁴⁰ or 10⁻¹¹ M, or any range or value therein Affinity and/or avidity measurements can be measured by KINEXA^{TIv1} and/or BIACOR™.

One embodiment of the invention includes isolated antibodies, or fragments of those antibodies, that bind to αVβ3 and αVβ5 integrins. As known in the art, the antibodies can be, for example, polyclonal, oligoclonal, monoclonal, chimeric, humanized, and/or fully human antibodies. Embodiments of the invention described herein also provide cells for producing these antibodies.

It will be appreciated that embodiments of the invention are not limited to any particular form of an antibody or method of generation or production. For example, the anti αVβ3 and αVβ5 antibody of the invention may be a full-length antibody (e.g., having an intact human Fc region) or an antibody fragment (e.g., a Fab, Fab′, F(ab′)₂, Fv or Dab (Dabs are the smallest functional binding units of human antibodies). In addition, the antibody may be manufactured from a hybridoma that secretes the antibody, or from a recombinantly produced cell that has been transformed or transfected with a gene or genes encoding the antibody.

Other embodiments of the invention include isolated nucleic acid molecules encoding any of the targeted binding agents, antibodies or fragments thereof as described herein, vectors having isolated nucleic acid molecules encoding anti-αVβ3 and αVβ5 integrin antibodies or a host cell transformed with any of such nucleic acid molecules. In addition, one embodiment of the invention is a method of producing an anti-αVβ3 and αVβ5 antibody of the invention by culturing host cells under conditions wherein a nucleic acid molecule is expressed to produce the antibody followed by recovering the antibody. It should be realized that embodiments of the invention also include any nucleic acid molecule which encodes an antibody or fragment of an antibody of the invention including nucleic acid sequences optimized for increasing yields of antibodies or fragments thereof when transfected into host cells for antibody production.

A further embodiment includes a method of producing high affinity antibodies to αVβ3 and αVβ5 integrins by immunizing a mammal with human αVβ3 and αVβ5 integrins, or a fragment thereof, and one or more orthologous sequences or fragments thereof.

In another embodiment, the invention includes an assay kit for binding to αVβ3 and αVβ5 integrins in mammalian tissues, cells, or body fluids to screen for kidney-related diseases. The kit includes a targeted binding agent or an antibody of the invention that binds to αVβ3 and αVβ5 integrins and a means for indicating the reaction of the antibody with αVβ3 and αVβ5 integrins, if present. In one embodiment, the antibody is a monoclonal antibody. In another embodiment, the antibody that binds αVβ3 and αVβ5 integrins is labeled. In still another embodiment the antibody is an unlabeled primary antibody and the kit further includes a means for detecting the primary antibody. In one embodiment, the means for detecting includes a labeled second antibody that is an anti-immunoglobulin. The antibody may be labeled with a marker selected from the group consisting of a fluorochrome, an enzyme, a radionuclide and a radiopaque material.

Other embodiments of the invention include pharmaceutical compositions having an effective amount of a targeted binding agent or an anti-αVβ3 and αVβ5 antibody of the invention in admixture with a pharmaceutically acceptable carrier or diluent. In yet other embodiments, the targeted binding agent or anti-αVβ3 and αVβ5 antibody of the invention, or a fragment thereof, is conjugated to a therapeutic agent. The therapeutic agent can be, for example, a toxin or a radioisotope.

Yet another embodiment includes methods for treating diseases or conditions associated with the uPAR mediated activation of αVβ3 and αVβ5 integrins in a patient, by administering to the patient an effective amount of a targeted binding agent or an anti-αVβ3 and anti-αVβ5 antibody of the invention. The targeted binding agent or anti-αVβ3 and anti-αVβ5 antibody of the invention can be administered alone, or can be administered in combination with additional antibodies or chemotherapeutic drug or radiation therapy. For example, a monoclonal, oligoclonal or polyclonal mixture of anti-αVβ3 and anti-αVβ5 antibodies can be administered in combination with a drug shown to inhibit a disease state or symptoms associated therewith. The method can be performed in vivo and the patient is preferably a human patient. In a preferred embodiment, the method concerns the treatment of kidney disease comprises: podocyte diseases or disorders, proteinuria, glomerular diseases, membranous glomerulonephritis, focal segmental glomerulonephritis, minimal change disease, nephrotic syndromes, pre-eclampsia, eclampsia, kidney lesions, collagen vascular diseases, stress, strenuous exercise, benign orthostatic (postural) proteinuria, focal segmental glomerulosclerosis (FSGS), IgA nephropathy, IgM nephropathy, membranoproliferative glomerulonephritis, membranous nephropathy, sarcoidosis, Alport's syndrome, diabetes mellitus, kidney damage due to drugs, Fabry's disease, infections, aminoaciduria, Fanconi syndrome, hypertensive nephrosclerosis, interstitial nephritis, Sickle cell disease, hemoglobinuria, multiple myeloma, myoglobinuria, diabetic nephropathy (DN), lupus nephritis, Wegener's Granulomatosis or Glycogen Storage Disease Type 1.

In some embodiments, the targeted binding agent(s) or anti-αVβ3 and anti-αVβ5 antibody(ies) of the invention is administered to a patient, followed by administration of a clearing agent to remove excess circulating antibody from the blood.

Nucleic Acid-based Agents: Nucleic acid-based agents such as antisense molecules and ribozymes can be utilized to target both the introns and exons of the αVβ3 and αVβ5 genes as well as at the RNA level to inhibit gene expression thereof, thereby inhibiting the activity of the uPAR mediated activation of these molecules. Further, triple helix molecules may also be utilized in inhibiting the αVβ3 and αVβ5 gene expression. Such molecules may be designed to reduce or inhibit either the wild type αVβ3 and αVβ5 gene, or if appropriate, the mutant αVβ3 and αVβ5 gene. Techniques for the production and use of such molecules are well known to those of skill in the art, and are succinctly described below.

In another preferred embodiment, αVβ3 and αVβ5 genes are modulated by targeting nucleic acid sequences involved in the expression and/or activity of αVβ3 and αVβ5 molecules. For example, regulatory regions would be a target to decrease the expression of αVβ3 and αVβ5 or the regions which encode for the signaling domains.

Antisense RNA and DNA molecules act to directly block the translation of mRNA by hybridizing to targeted mRNA and preventing protein translation. Antisense approaches involve the design of oligonucleotides that are complementary to a target gene mRNA. The antisense oligonucleotides will bind to the complementary target gene mRNA transcripts and prevent translation. Absolute complementarity, although preferred, is not required.

A sequence “complementary” to a portion of an RNA, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have been shown to be effective at inhibiting translation of mRNAs as well. Wagner (1994) Nature 372:333-335. For example, oligonucleotides complementary to either the 5′- or 3′-untranslated, non-coding regions of the human or mouse gene of urokinase receptor molecules could be used in an antisense approach to inhibit translation of endogenous urokinase receptor molecules mRNA.

In another preferred embodiment, the antisense approach can be used to target negative regulators of αVβ3 and αVβ5 expression and/or function.

Oligonucleotides complementary to the 5′ untranslated region of the mRNA should include the complement of the AUG start codon. Antisense oligonucleotides complementary to mRNA coding regions are less efficient inhibitors of translation but could be used in accordance with the invention. Whether designed to hybridize to the 5′-, 3′- or coding region of target gene mRNA, antisense nucleic acids are preferably at least six nucleotides in length, and are more preferably oligonucleotides ranging from 6 to about 50 nucleotides in length. In specific aspects the oligonucleotide is at least 10 nucleotides, preferably at least 17 nucleotides, more preferably at least 25 nucleotides and most preferably at least 50 nucleotides.

Alternatively, antisense molecules may be designed to target the translated region, i.e., the cDNA of the αVβ3 and αVβ5 genes. For example, the antisense RNA molecules targeting the full coding sequence or a portion of the mature murine urokinase receptor molecules (Kirschke et al. (2000) Euro. J. Cancer 36:787-795) may be utilized to inhibit expression of urokinase receptor molecules and thus reduce the activity of its enzymatic activity. In addition, a full length or partial urokinase receptor molecules cDNA can be subcloned into a pcDNA-3 expression vector in reversed orientation and such a construct can be transfected into cells to produce antisense polyRNA to block endogenous transcripts of a uPAR, such as urokinase receptor molecules, and thus inhibit uPAR's expression.

In vitro studies may be performed to quantitate the ability of the antisense oligonucleotide to inhibit gene expression. It is preferred that these studies utilize controls that distinguish between antisense gene inhibition and nonspecific biological effects of oligonucleotides. It is also preferred that these studies compare levels of the target RNA or protein with that of an internal control RNA or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides, or agents facilitating transport across the cell membrane (See, e.g., Letsinger (1989) Proc. Natl. Acad. Sci. U.S.A. 86:6553-6556) or the blood-brain barrier, hybridization-triggered cleavage agents. See, e.g., Krol (1988) Bio Techniques 6:958-976 or intercalating agents. See, e.g., Zon (1988) Pharm. Res. 5:539-549. The oligonucleotide may be conjugated to another molecule, e.g., a peptide, hybridization triggered cross-linking agent, transport agent, hybridization-triggered cleavage agent, etc.

The antisense oligonucleotide may comprise at least one modified base moiety which is selected from the group consisting of, but not being limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomet-hyluracil, dihydrouracil,13-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, β-D-mannosylqueosine, 5′-methoxycarboxymethyluracil, 5-methoxyuracil, 2-methylthio-N6-isopenten-yladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group consisting of, but not being limited to arabinose, 2-fluoroarabinose, xylulose, and hexose.

In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

Ribozyme molecules designed to catalytically cleave target gene mRNA transcripts can also be used to prevent translation of target gene mRNA and, therefore, expression of target gene product. See, e.g. Sarver et al. (1990) Science 247:1222-1225.

Ribozymes are enzymatic RNA molecules capable of catalyzing the specific cleavage of RNA. The mechanism of ribozyme action involves sequence specific hybridization of the ribozyme molecule to complementary target RNA, followed by an endonucleolytic cleavage event. The composition of ribozyme molecules should include one or more sequences complementary to the target gene mRNA, and should include the well known catalytic sequence responsible for mRNA cleavage.

While ribozymes that cleave mRNA at site specific recognition sequences can be used to destroy target gene mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art.

Endogenous αVβ3 and αVβ5 gene expression can also be reduced by inactivating or “knocking out” the targeted αVβ3 and αVβ5 genes or their promoters using targeted homologous recombination. Smithies et al. (1985) Nature 317:230-234; Thomas and Capecchi, (1987) Cell 51:503-512; and Thompson et al. (1989) Cell 5:313-321.

Alternatively, endogenous αVβ3 and αVβ5 gene expression can be reduced by targeting deoxyribonucleotide sequences complementary to the regulatory region of the αVβ3 and αVβ5 genes (i.e., the target gene promoter and/or enhancers) to form triple helical structures that prevent transcription of the target gene in target cells in the body. See generally, Helene (1991) Anticancer Drug Des. 6:569-584; Helene et al. (1992) Ann. N.Y. Acad. Sci. 660:27-36; and Maher (1992) Bioassays 14:807-815.

Nucleic acid molecules to be used in triplex helix formation for the inhibition of transcription should be single stranded and composed of deoxynucleotides. The base composition of these oligonucleotides must be designed to promote triple helix formation via Ho6gsteen base pairing rules, which generally require sizeable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, contain a stretch of G residues. These molecules will form a triple helix with a DNA duplex that is rich in GC pairs, in which the majority of the purine residues are located on a single strand of the targeted duplex, resulting in GGC triplets across the three strands in the triplex.

### Administration of Compositions to Patients

The compositions or agents identified by the methods described herein may be administered to animals including human beings in any suitable formulation. For example, the compositions for modulating protein degradation may be formulated in pharmaceutically acceptable carriers or diluents such as physiological saline or a buffered salt solution. Suitable carriers and diluents can be selected on the basis of mode and route of administration and standard pharmaceutical practice. A description of exemplary pharmaceutically acceptable carriers and diluents, as well as pharmaceutical formulations, can be found in Remington's Pharmaceutical Sciences, a standard text in this field, and in USP/NF. Other substances may be added to the compositions to stabilize and/or preserve the compositions.

The compositions of the invention may be administered to animals by any conventional technique. The compositions may be administered directly to a target site by, for example, surgical delivery to an internal or external target site, or by catheter to a site accessible by a blood vessel. Other methods of delivery, e.g., liposomal delivery or diffusion from a device impregnated with the composition, are known in the art. The compositions may be administered in a single bolus, multiple injections, or by continuous infusion (e.g., intravenously). For parenteral administration, the compositions are preferably formulated in a sterilized pyrogen-free form.

The compounds can be administered with one or more therapies. The chemotherapeutic agents may be administered under a metronomic regimen. As used herein, “metronomic” therapy refers to the administration of continuous low-doses of a therapeutic agent.

Dosage, toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of a compound (i.e., an effective dosage) means an amount sufficient to produce a therapeutically (e.g., clinically) desirable result. The compositions can be administered one from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compounds of the invention can include a single treatment or a series of treatments.

### Reduction of Proteinuria

In one aspect, the invention includes a method for reducing proteinuria or urinary albumin in a subject. In this method, the subject is administered a sufficient amount of an agent that targets and modulates the function of αVβ3 and/or αVβ5 integrins such that proteinuria or concentrations of urinary albumin are reduced by at least 10, 20, 30, 40, 50, 60, 70, 80, 90 or more percent post-treatment. The agent can be a monoclonal antibody that specifically binds αVβ3 and/or αVβ5 integrins (e.g., CNTO 95). Alternatively, the agent can inhibit uPARand/or suPAR binding to the urokinase receptor or can specifically bind to these molecules thus preventing their binding or deposition in the podocytes.

Treatment of Subjects with Abnormal suPAR levels: The invention includes a method including a step of administering an agent that targets and modulates the function of αVβ3 and/or αVβ5 integrins to a subject having abnormally high serum suPAR (e.g., greater than 3500, 3600, 3700, 3800, 390, 4000, 4500, or 5000 pg/ml serum as determined by ELISA or other assays). In one embodiment, the agent is a monoclonal antibody that specifically binds αVβ3 and/or αVβ5 integrins (e.g., CNTO 95). This method can also include a step of determining whether the subject has an abnormal serum suPAR level (with or without renal disease or symptoms) and/or a step of selecting and/or modulating the dosing of the agent that targets and modulates the function of αVβ3 and/or αVβ5 integrins according to the subject's suPAR levels (e.g., lower dose for patients with lower but still high suPAR levels, and titrating the dose according to a subject's response).

The invention also includes a method for reducing pathologic levels of activated αVβ3 and/or αVβ5 integrins on human podocytes by contacting the cells with an agent that targets and modulates the function of αVβ3 and/or αVβ5 integrins such as CNTO 95.

In another aspect, the invention features a method including the step of administering an agent that targets and modulates the function of αVβ3 and/or αVβ5 integrins such as CNTO 95 or a like antibody to a subject with proteinuria but not cancer and/or a subject that is also being treated with other drugs for kidney disease (e.g., ACE inhibitors, angiotensin receptor blockers, diuretics, steroids, calcium carbonate, calcitriol, sevelamer, erythropoietin, darbepoetin, iron, and/or vitamin D) or for drugs that can address any possible side effects of CNTO 95 (e.g., acetaminophen, ibuprofen, or other pain or fever reducers; antihistamines; and/or anti-nausea medications). The step of administering an agent that targets and modulates the function of αVβ3 and/or αVβ5 integrins may be performed by the methods described herein as well as by more specifically directing the kidney using a renal infusion system such as the BENEPHIT® system (AngioDynamics).

Reduction of Activated αVβ3 on Podocytes: Also within the invention is a method of reducing the function of activated αVβ3 on podocytes, e.g., in a subject with abnormally high levels of activated a Vf33 on his/her podocytes (e.g., more than 100, 200, 300, 400, or 500% of normal levels). This method can include the step of contacting the podocytes with an agent that targets and modulates the function of αVβ3 and/or αVβ5 integrins such as CNTO 95.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments.

All documents mentioned herein are incorporated herein by reference. All publications and patent documents cited in this application are incorporated by reference for all purposes to the same extent as if each individual publication or patent document were so individually denoted. By their citation of various references in this document, Applicants do not admit any particular reference is “prior art” to their invention.

What is claimed is:

1. A method of reducing proteinuria in a subject, the method comprising the step of administering to the subject an amount of a pharmaceutical composition comprising an antibody that specifically binds αVβ3 integrin and/or αVβ5 integrin, in therapeutically effective amounts to reduce proteinuria in a subject.

2. The method of claim 1, wherein the antibody is a monoclonal or polyclonal antibody.

3. The method of claim 1, wherein the antibody is a human antibody, a humanized antibody, or fragments thereof.

4. The method of claim 1, wherein the antibody is CNTO 95.

5. The method of claim 2, wherein the proteinuria in a patient is reduced by at least about 20% as measured by the patient's urinary protein concentrations.

6. The method of claim 2, wherein the subject suffers from focal segmental glomerulosclerosis.

7. A method comprising the steps of
(a) identifying a subject with proteinuria;
(b) administering to the subject a pharmaceutical composition comprising a monoclonal antibody that specifically binds αVβ3 integrin; and then
(c) analyzing urinary protein concentration in the subject.

8. The method of claim 7, further comprising the step of:
re-administering to the subject the pharmaceutical composition.

9. The method of claim 7, wherein the antibody is a human or humanized antibody.

10. The method of claim 7, wherein the antibody is CNTO 95.

11. The method of claim 7, wherein the proteinuria is reduced by at least 20% after the step of administering the pharmaceutical composition.

12. The method of claim 7, wherein the subject suffers from focal segmental glomerulosclerosis.

13. A method comprising the steps of:
(a) identifying a subject with serum suPAR levels greater than 3000 pg/ml; and
(b) administering to the subject a pharmaceutical composition comprising a monoclonal antibody the specifically binds a αVβ3 integrin.

14. The method of claim 13, further comprising the step of:
re-administering to the subject the pharmaceutical composition.

15. The method of claim 13, wherein the antibody is a human or a humanized antibody.

16. The method of claim 13, wherein the antibody is CNTO 95.

17. The method of claim 13, wherein the subject suffers from focal segmental glomerulosclerosis or any other glomerular disease before and after renal transplantation.

18. The method of claim 13, further comprising the step (c) of analyzing urinary protein concentration in the subject after the step of administering the pharmaceutical composition.

19. The method of claim 13, wherein the urinary protein concentration in the subject is reduced by at least 20% after the step of administering the pharmaceutical composition.

20. A pharmaceutical composition comprising an agent which specifically modulates alphaV (αV) integrin expression, function, signaling or combinations thereof in vivo.

21. The pharmaceutical composition of claim 20, wherein the agent comprises an antibody, aptamer, small molecule, enzyme, oligonucleotide, polynucleotide, peptide, cyclic peptides, polypeptide, carbohydrate, glycosylated carbohydrate, synthetic molecule, organic or inorganic molecule.

22. The pharmaceutical composition of claim 20, wherein the agent specifically binds to alphaVbeta3 (αVβ3) and/or alphaVbeta5 (αVβ5) integrins.

23. The pharmaceutical composition of claim 20, wherein the agent is an antibody.

24. A method of preventing or treating kidney disease or disorders in vivo, comprising administering to a patient an agent in a therapeutically effective amount, whereby the agent modulates the expression, function or signaling of alphaV integrin molecules in vivo as measured by a decrease in the patient's urinary protein concentration; and, preventing or treating kidney disease in vivo.

25. The method of claim 24, wherein the agent comprises an antibody, aptamer, small molecule, enzyme, oligonucleotide, polynucleotide, peptide, polypeptide, synthetic molecule, organic or inorganic molecule.

26. The method of claim 24, wherein the agent specifically binds to alphaVbeta3 (αVβ3) and/or alphaVbeta5 (αVβ5) integrins.

27. The method of claim 24, wherein the agent specifically binds to urokinase receptor molecules (uPAR) or fragments thereof, soluble urokinase receptor molecules (suPAR) or fragments thereof, or combinations thereof.

28. The method of claim 24, wherein the kidney disease or disorders comprise: podocyte diseases or disorders, proteinuria, glomerular diseases, progressive glomerular disease membranous glomerulonephritis, focal segmental glomerulonephritis, minimal change disease, nephrotic syndromes, pre-eclampsia, eclampsia, kidney lesions, collagen vascular diseases, stress, strenuous exercise, benign orthostatic (postural) proteinuria, focal segmental glomerulosclerosis (FSGS), IgA nephropathy, IgM nephropathy, membranoproliferative glomerulonephritis, membranous nephropathy, sarcoidosis, Alport's syndrome, diabetes mellitus, kidney damage due to drugs, Fabry's disease, infections, aminoaciduria, Fanconi syndrome, hypertensive nephrosclerosis, interstitial nephritis, Sickle cell disease, hemoglobinuria, multiple myeloma, myoglobinuria, diabetic nephropathy (DN), lupus nephritis, Wegener's Granulomatosis or Glycogen Storage Disease Type 1.

29. The method of claim 28, wherein the kidney disease or disorder is proteinuria.

30. The method of claim 28, wherein the kidney disease or disorder is glomerular disease.

31. A method of treating subjects with abnormal urokinase receptor molecules (uPAR) or soluble urokinase receptor molecules (suPAR) levels comprising the steps of:
(a) identifying a subject with serum suPAR levels about greater than 3000 ng/ml; and
(b) administering to the subject a pharmaceutical composition comprising an agent that specifically binds alphaVbeta3 (αVβ3) and/or alphaVbeta5 (αVβ5) integrin.

32. The method of claim 31, further comprising the step of: re-administering to the subject the pharmaceutical composition.

33. The method of claim 31, wherein the agent is an antibody, or a fragments thereof.

34. The method of claim 31, wherein the urinary protein concentration in the subject is reduced by at least 20% as compared to a normal control after the step of administering the pharmaceutical composition.
